(19) United States
(12) Patent Application Publication
(10) Pub. No.: US 2018/0193398 A1
(43) Pub. Date: Jul. 12, 2018

(54) Prevention of symptoms related to fluctuating female hormone levels

(71) Applicant: Jennifer Marie Johnson, Los Angeles, CA (US)

(72) Inventor: Jennifer Marie Johnson, Los Angeles, CA (US)

(21) Appl. No.: 15/732,451

(22) Filed: Nov. 13, 2017

Related U.S. Application Data
(60) Provisional application No. 62/497,258, filed on Nov. 14, 2016.

(51) Int. Cl. A61K 36/65; A61K 31/593; A61K 31/122; A61K 31/4415; A61K 31/519; A61K 31/714; A61K 33/06; A61K 33/22; A61K 35/745; A61K 35/744; A61K 35/747; A61K 31/51; A61K 31/525; A61K 31/455; A61K 31/4188; A61K 31/197; A61K 36/48; A61K 36/03; A61K 36/05; A61K 36/04; A61K 31/202; A61K 31/375; A61K 33/26; A61K 33/04; A61K 33/30; A61K 31/355; A61K 36/53; A61K 31/198; A61K 36/185; A61K 36/484; A61K 31/385; A61P 5/24

(52) U.S. Cl. CPC A61K 36/65; A61P 5/24; A61K 31/122; A61K 31/4415; A61K 31/519; A61K 31/714; A61K 33/06; A61K 33/22; A61K 35/745; A61K 35/744; A61K 35/747; A61K 31/51; A61K 31/525; A61K 31/455; A61K 31/4188; A61K 31/197; A61K 36/48; A61K 36/03; A61K 36/05; A61K 36/04; A61K 31/202; A61K 31/375; A61K 33/26; A61K 33/04; A61K 33/30; A61K 31/355; A61K 36/53; A61K 31/198; A61K 36/185; A61K 36/484; A61K 31/385; A61K 31/593

(57) ABSTRACT

PMS is a catch-all phrase that can include over 150 physical and emotional symptoms affecting over eighty percent of women during their lifetimes. The invention herein of a hormone balancing regimen, both method and system, is intended to prevent symptoms related to fluctuating hormone levels. The regimen provides essential nutrients that coincide with the female cycle to mitigate and eliminate unwanted, unnecessary, extraneous, and negative symptoms. Additionally, while it does not necessarily is not promising to fix underlying conditions, it does provide absorbable, non-toxic vitamins and minerals that the female body needs and increases overall health, including improvements in sleep, mood, and vitality throughout the cycle, not just for the pre-menses phase.

### REFERENCE TO RELATED APPLICATIONS

This application takes priority from provisional application Ser. No. 62/497,258 filed on Nov. 14, 2016.

### BACKGROUND OF THE PRESENT DISCLOSURE

### Field of the Invention

Present Disclosure relates generally to biological systems, and, more particularly, to providing a method and system by which the hormone levels of a female can be maintained at a level that provides physical and mental equanimity.

Currently women are taken out month after month with cramps, bloating, emotional rage, sadness, depression, acne, headaches, breast pain, fatigue, cravings, low self-confidence, excessive weight gain, amongst other symptoms. What this creates is amazing women not being powerful or accomplishing their dreams or living fulfilled lives. The low self-confidence is sometimes hard for women to build up as their actions and emotions that were expressed during their premenstrual week are damaging to their personal and business relationships. They are then having to apologize for their behavior and still not gaining full respect of those they impacted. This is especially true when they repeat the same behaviors the following months.

These premenstrual symptoms occur because the women's hormonal system is very complex. However, although complex the women's hormonal system is on a routine cycle. The cycle is generally 28 days in length and follows the pattern outlined in the photo below in terms of egg cycle, hormone levels, etc.

The Jenius Cycle vitamin pack is the solution to this problem because it nurtures the women's natural cycle with the vitamins it needs at the right times throughout the month to work with and manage the fluctuating hormone levels.

Although there are many PMS relief products on the market there is not an easy to take daily, monthly vitamin pack that follows the cycle to pinpoint the exact times that each mineral and vitamin is necessary for proper managing. The current products create confusion amongst consumers as well as over taking minerals and vitamins throughout the entire month, which is not necessary and problematic when increasing estrogen or progesterone levels when they are already high. There are several “hormonal balancing experts” that tell you what nutrients to eat/take at the proper times of the female cycle. There is just not this simple solution that has been created by the Jenius Cycle. Jenius Cycle contains all highly absorbable with no added fillers or contradictory/harmful ingredients. The founders are very passionate about quality and will not compromise for price or ease.

### SUMMARY OF THE PRESENT DISCLOSURE

Accordingly, there is provided an improved method by which the negative symptoms of the female cycle is mitigated by working with fluctuating hormone levels throughout the four phases of the menstrual cycle.

### BRIEF DESCRIPTION OF THE FIGURES

The organization and manner of the structure and operation of the Present Disclosure, together with further objects and advantages thereof, may best be understood by reference to the following Detailed Description, taken in connection with the accompanying Figures, wherein like reference numerals identify like elements, and in which:

FIG. 1 illustrates a preferred embodiment of the basic formula which provides an example of the method. The preferred embodiment formula is flexible and can vary based on age and significant differences that affect a person's health. The formula further varies day by day to coincide with the female cycle. The preferred formula has optional additions. As well as substitutions with vitamins and/or minerals that produce the same result. The days listed are approximate and vary based on a 28-day cycle and can still be varied based on an individual females personal cycle including phase lengths. It can be clearly seen in FIG. 1 that this is a female-cycle based daily and nightly method that coincides with the body's needs and thereby mitigates and eliminates symptoms often related with PMS, usually experienced by women the week or two prior to their menses (their period.) It can further be seen that such a method will also improve a females overall health, sleep, mood, and vitality throughout the month, not just for the pre-menses phase. Females might notice the difference more in the time prior to their menses due to having lowest amounts of estrogen and progesterone in the body and being able to experience the symptoms to a greater degree.

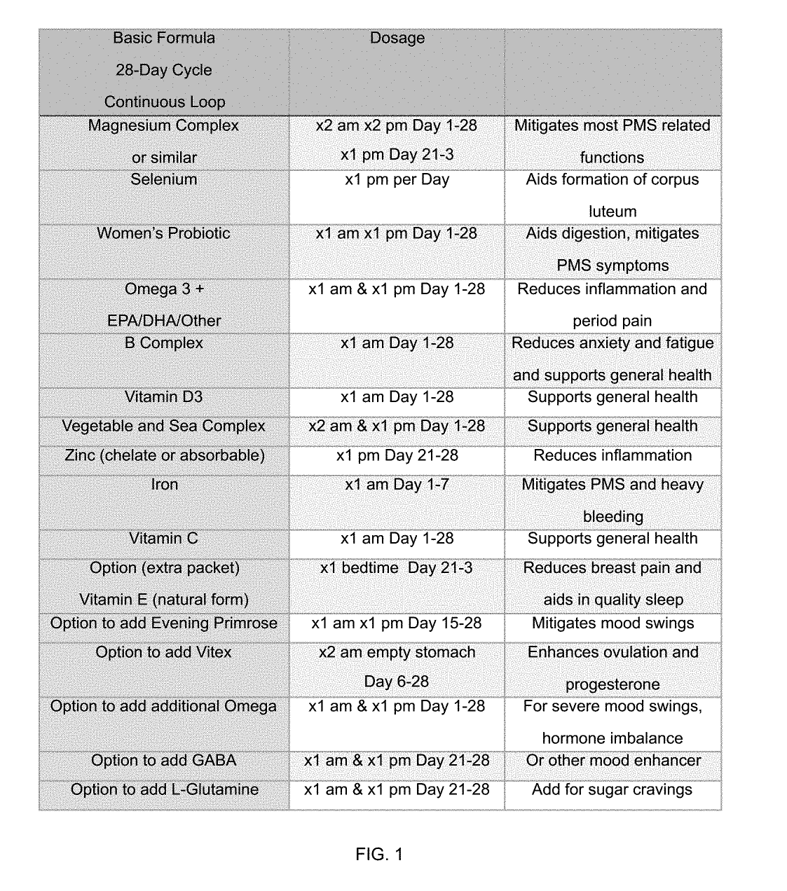

FIG. 2 illustrates the addition of the pre-AM packet for special cases including missed periods. Taken Days 1-24 and then not day 25-28 or if do have period then take day 5-28. The Day 1 is the first day of menstruation, this is the first day that there is a steady flow of blood. To be taken first thing in the morning on an empty stomach, this is when the pituitary gland is most receptive and will be the most effective. It can be seen that the addition of vitex, also known as chasteberry, has the medicinal ability to positively affect hormonal health. It is derived from dopaminergic compounds present in the herb that work with the hypothalamus and pituitary glands. The addition of vitex for present invention is that it increases luteinizing hormone, modulates prolactin and aids in the inhibition of the release of follicle-stimulating hormone, which all aid in balancing the ratio of progesterone to estrogen, slightly raising the levels of progesterone. Vitex is an herb that helps the body raise its own levels of progesterone and the added supplementation can help to modulate prolactin and get a healthy balance of estrogen and progesterone back on track. In other words, the addition of vitex assists a return of normal functioning for those who are experiencing hypothalamic amenorrhea (not having a period) and is also an antidepressant.

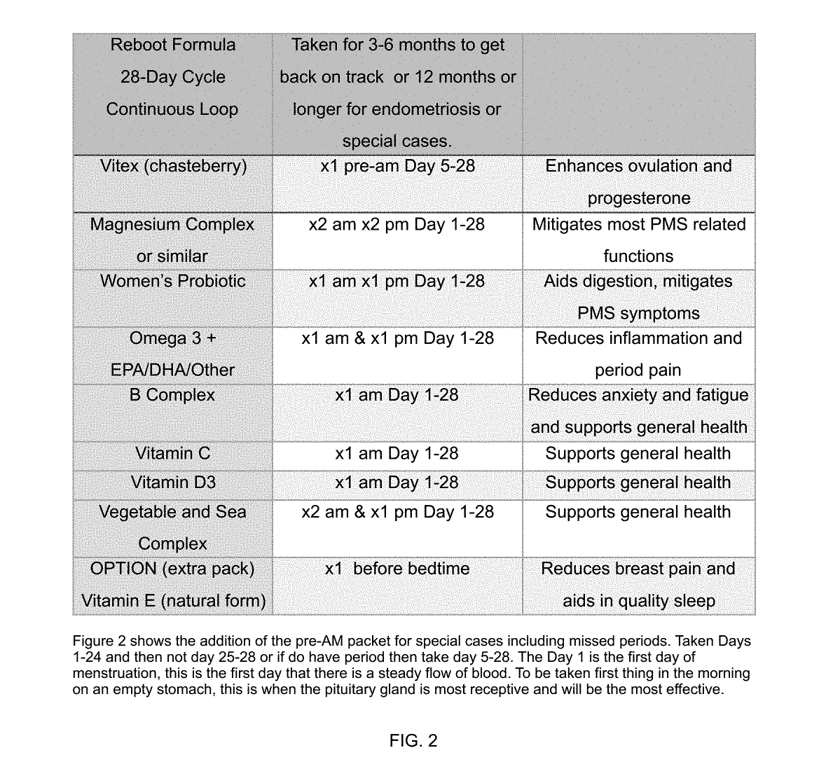

FIG. 3 illustrates the components of a magnesium complex, one of the complexes used in the present invention. The present configuration of this complex is one of many that fit within the spirit and scope of this patent.

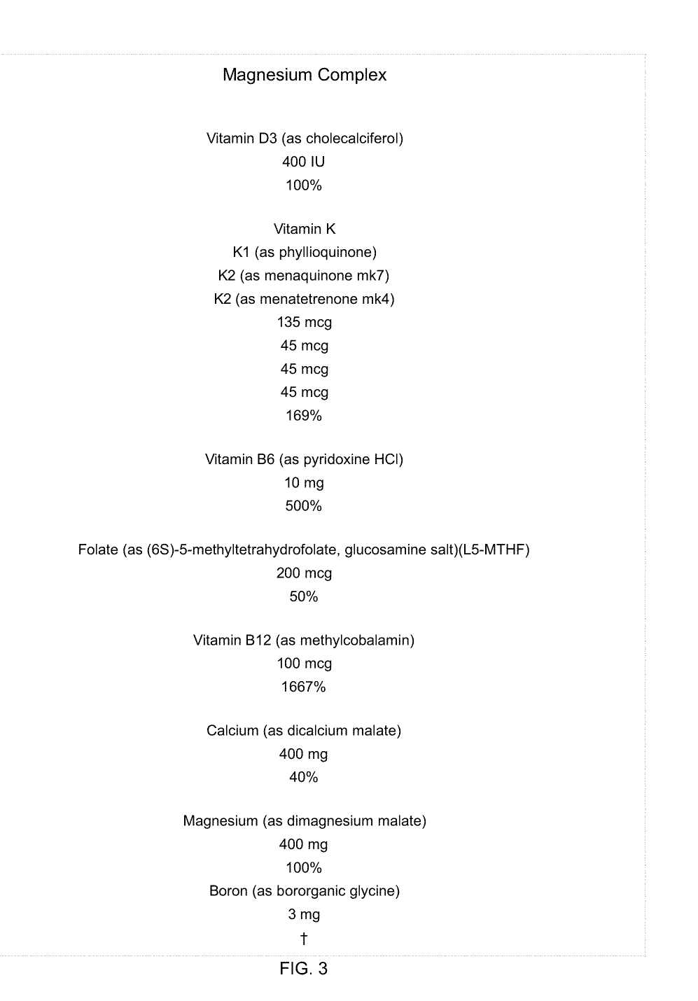

FIG. 4 illustrates the components of a B complex, one of the complexes used in the present invention, which is helpful in aiding HPA axis dysfunction, eliminating anxiety, and eliminating fatigue. Additionally, Vitamin B6 has an impactful role as it normalizes the metabolism of biogenic amines by aiding the production of various pyridoxal phosphate dependent enzymatic reactions.

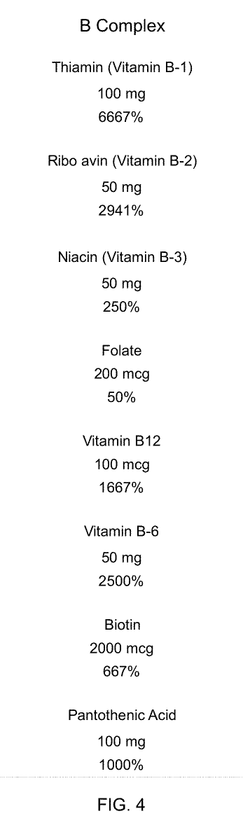

FIG. 5 illustrates the components of a vegetable and sea complex, one of the complexes used in the present invention. The vegetable and sea complex of the present invention preferably contains organic green, brown and red sea vegetables with a healthful serving of alfalfa leaves. This rich diversity of nutrients promote healthy digestion and elimination, support healthy cholesterol levels, pH balance, bone, joint function, skin, hair, nails, cardiovascular function, glands, organs, and immune system, inhibit the uptake of heavy metals, remove toxins and heavy metals, protect against radioactive iodine. Also, the organic iodine will promote healthy thyroid function for improved metabolism and weight control. All are important for maintaining overall health and hormone balance.

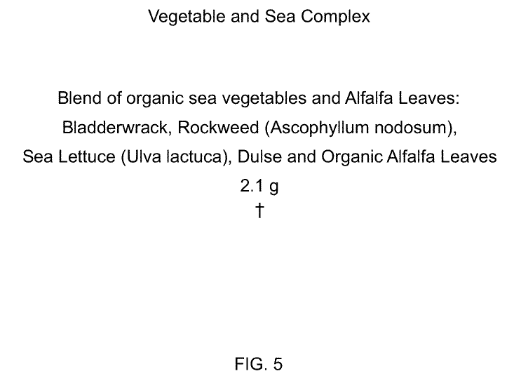

FIG. 6 illustrates the exemplar components of a probiotic designed for women, used in the present invention. Probiotics, including strains that aid in the gastrointestinal tract, vagina, and can survive low pH balance environments, and thus play an important role in hormonal balance, which leads to elimination of PMS. Probiotics are important to consume consistently throughout the month to maintain a healthy stomach biome to support immune function. It is important to have a healthy stomach biome to enable proper vitamin and mineral absorption. If the stomach is acidic or unstable it can prevent absorption by breaking down the vitamins and minerals in the stomach acid and not in the small and large intestines, which is where they are intended to be absorbed into the bloodstream. There are many strains that fit a profile for women's health, and an exemplar of one that fits within the spirit and scope of the present invention is included in FIG. 6.

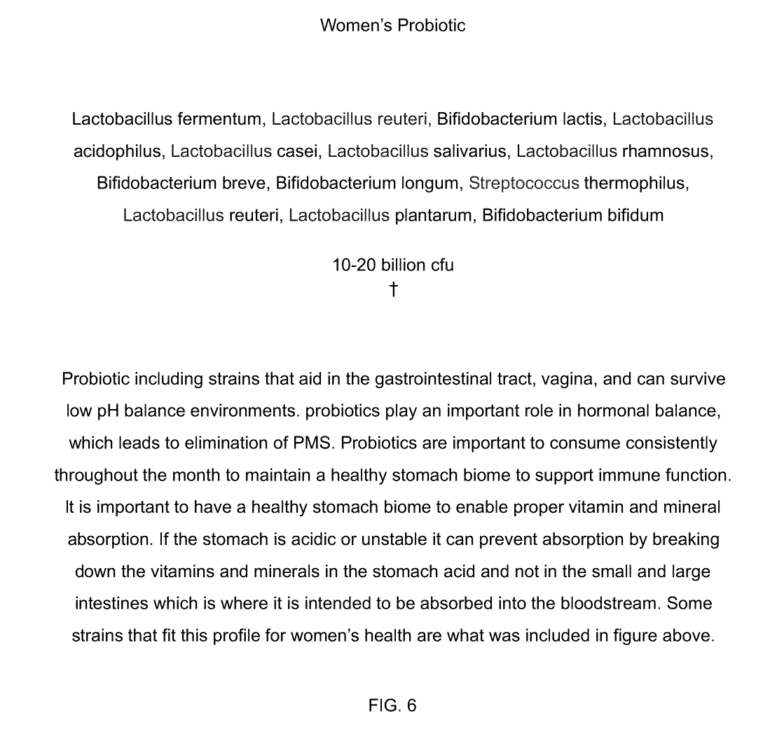

### DESCRIPTION OF THE PREFERRED EMBODIMENTS

While the Present Disclosure may be susceptible to embodiment in different forms, there is shown in the Figures, and will be described herein in detail, specific embodiments, with the understanding that the Present Disclosure is to e considered and exemplification of the principles of the Present Disclosure, and is not intended to limit the Present Disclosure to that as illustrated.

As such, references to feature or aspect are intended to describe a feature or aspect of an example of the Present Disclosure, not to imply that every embodiment thereof must have the described feature or aspect. Furthermore, it should be noted that the description illustrates a number of features. While certain features have been combined together to illustrate potential system designs, those features may also be used in other combinations not expressly disclosed. Thus, the depicted combinations are not intended to be limiting, unless otherwise noted.

In the embodiments illustrated in the Figures, representations of direction such as up, down, left, right, front and rear, used for explaining the structure and movement of the various elements of the Present Disclosure, are not absolute, but relative. These representations are appropriate when the elements are in the position shown in the Figures. If the description of the opposition of the elements changes, however, these representations are to be changed accordingly.

The vitamin and mineral menstrual cycle system contains daily vitamins and minerals that aid in nurturing body to alleviate hormone changes and replenish vitamins and minerals that are used therefore being depleted from each of the cycles use. Use of the Jenius Cycle system is a vitamin and mineral system that coincides with the female menstrual cycles predictable monthly cycle. Use not only aids in reducing symptoms associated with PMS, generally the week before menstruation occurs as well as during menstruation which was its primary reason for creation, it has shown to keep women more healthy and happy. Better full night sleep, ameliorated anxiety, improved self-confidence, healthy daily bowel movements, and increased energy and sex drive were the most reported additional benefits. It is believed that this is due to the lack of essential nutrients that women are intaking through diet, other supplements not being absorbable forms, and depletion of basic minerals and vitamins that the body needs to perform designated functions through chronic internal and external stressors, environmental toxins, and other depletion causes.

The Jenius Cycle not only contains high quality bioavailable, absorbable vitamins and minerals it also is easier for women to take which equals better results. Women now are working and don't have time to open multiple bottles of vitamins and keep track of what part of their cycle they're at to decipher which supplements are needed to coincide with their bodies needs.

The Jenius Cycle consists of a roll or daily packets of vitamins that contain an am and a pm pack of vitamins for daily consumption that change to coincide with the female cycle to support the endocrine system resulting in hormone balance.

Day 1-4 have added iron, zinc, magnesium, calcium, k1, k2, boron and other trace minerals that replenish minerals that are depleted from the extra blood and excretion which takes place during the menstruation phase of the cycle. This results in relieving cramps immediately as well as keeping body healthy as to not cause damage for long-term health, depletion of iron often leads anemia and more debilitating and dangerous menstruations.

Day 5-13 have the baseline vitamins that are essential to maintain a healthy endocrine system.

Day 14-28 have added Vitamin B6 and C to ward off cravings, and to aid in hormone elimination of free radical damage.

Day 21-28 have added mood stabilizer, pharma GABA or similar effect supplement to keep mind stable.

Day 25-28 have added zinc for prevent breakouts because zinc kills acne causing bacteria. Zinc also reduces inflammatory response to bacteria. Added magnesium, and calcium for mood stabilization, pre-muscle cramp, sugar cravings, dizziness and headache prevention.

Detailed vitamin breakdown by daily packets:

Day 1-28 Healthful-Bone (Calcium, magnesium, boron, vitamin K complex)

Day 1-28 Vitamin D3

Day 1-28 Healthful Greens & Reds

Day 1-28 Women's Probiotic

Day 1-28 Omega 3's

Day 1-28 B complex

Day 1-28 Vitamin C

Day 25-4 Zinc

Day 1-4 added K1, K2, boron

Day 1-4 added magnesium

Day 1-4 Iron

Day 14-28 added Vitamin C

Day 14-28 added Vitamin B6

Day 21-28 added mood stabilizer

Finally, while a preferred embodiment of the Present Disclosure is shown and described, it is envisioned that those skilled in the art may devise various modifications without departing from the spirit and scope of the foregoing Description and the appended Claims.

What is claimed is:

1. A method of preventing symptoms related to fluctuating female hormone levels by nurturing the body in order to mitigate and potentially eliminate the wide range of PMS symptoms and improve a woman's overall health by intaking a sequence of vitamins and minerals in a very particular order and timed to coincide with the woman's 28-day menstrual cycle, said cycle having approximately four phases, the method comprising:
a. intaking, on day 1 through day 7, phase 1, of a woman's menstrual cycle, a magnesium complex, a woman's probiotic, a B complex, a vegetable and sea complex, omega 3, vitamin C, vitamin D3, iron and selenium;
b. intaking, on day 8 through day 20, phase 2 and 3, of a woman's menstrual cycle, a magnesium complex, a woman's probiotic, a B complex, a vegetable and sea complex, omega 3, vitamin C, vitamin D3, and selenium;
c. intaking, on day 21 through day 28, phase 4, of a woman's menstrual cycle, a magnesium complex, a woman's probiotic, a B complex, a vegetable and sea complex, omega 3, vitamin C, vitamin D3, selenium, zinc, and additional magnesium complex; and
d. intaking, days 1-3 and days 21-28 of a woman's menstrual cycle, an additional dose of magnesium complex.

2. The method of prevention of symptoms related to fluctuating female hormone levels defined in claim 1 further including intaking on days 1-3 and days 21-28 of a woman's menstrual cycle, a dose of vitamin E.

3. The method of prevention of symptoms related to fluctuating female hormone levels defined in claim 1 further including intaking on days 6 through 28 of a woman's menstrual cycle, a dose of vitex.

4. The method of prevention of symptoms related to fluctuating female hormone levels defined in claim 1 further including intaking on days 1 through 28 of a woman's menstrual cycle, a dose of omega.

5. The method of prevention of symptoms related to fluctuating female hormone levels defined in claim 1 further including intaking on days 21 through 28 of a woman's menstrual cycle, a dose of GABA.

6. The method of prevention of symptoms related to fluctuating female hormone levels defined in claim 1 further including intaking on days 21 through 28 of a woman's menstrual cycle, a dose of L-Glutamine.

7. The method of prevention of symptoms related to fluctuating female hormone levels defined in claim 1 further including intaking on days 15 through 28 of a woman's menstrual cycle, a dose of evening primrose.

8. The method of prevention of symptoms related to fluctuating female hormone levels defined in claim 1 wherein the intaking further comprises administering the doses in conventional forms including capsules, tablets, powders, elixirs, as additives to food or beverages, transdermally, injected, inhaled, or any form that may currently be unknown.

9. The method of prevention of symptoms related to fluctuating female hormone levels defined in claim 1 wherein the vitamin and mineral regimen coincides with the female cycle to provide nutrients that correlate with needs of the womens body, and additionally is flexible to meet an individual women's cycle, usually ranging 24-35 days, by adding or subtracting a day from each phase to match the individual menstrual cycle.

10. The method of prevention of symptoms related to fluctuating female hormone levels defined in claim 1 wherein, if a woman becomes symptomatic earlier than one week prior to the start of the menses, the method moves to the step at phase 4, and wherein phase 4 is continues for two weeks before restarting the basic method at phase 1.

11. The method defined in claim 1, further comprising the steps of intaking other consumables, performing certain actions, exercises and affirmations.

12. The system of preventing symptoms related to fluctuating female mammalian hormone levels by nurturing the body in order to mitigate and potentially eliminate the wide range of PMS symptoms and improve a female's overall health by intaking a sequence of vitamins and minerals timed to coincide with a female mammalian menstrual cycle, the system comprising prepacked vitamins and minerals prepared for each day that contain specific vitamins for the phases of the female mammalian cycle to provide supplementation to eliminate and mitigate symptoms of premenstrual syndrome and general good working health of a female's internals.

13. The system of preventing symptoms defined in claim 12, wherein the prepacked vitamins and minerals are arranged in a roll pack and in day and time order for every day of the female's cycle.

14. The system of preventing symptoms defined in claim 12, wherein the prepacked vitamins and minerals are arranged in day and time order for every day of the female's cycle.

15. The system of preventing symptoms defined in claim 12, wherein the day and time ordered arrangement further includes reminders to intake other consumables, perform certain actions, exercises and affirmations.

16. A method to reduce the instance of irregular periods, PCOS or hypothalamic amenorrhea, the method comprising:
a. intaking, on day 1 through day 4 of the regimen, a magnesium complex, a women's probiotic, omega 3, B complex, vegetable and sea complex, vitamin C, and vitamin D; and
b. intaking, on day 5 through day 28 of the regimen, a magnesium complex, a women's probiotic, omega 3, B complex, vegetable and sea complex, vitamin C, and vitamin D, and vitex.

17. The method defined in claim 16, further comprising ingesting zinc.

18. The method defined in claim 16, further comprising ingesting peony and licorice.

19. The method defined in claim 16, further comprising ingesting alpha-lipoic acid.

20. The method defined in claim 16, further comprising the steps of intaking other consumables, performing certain actions, exercises and affirmations.
